(19) United States
(12) Patent Application Publication
(10) Pub. No.: US 2024/0341686 A1
(43) Pub. Date: Oct. 17, 2024

(54) AUGMENTED-REALITY SYSTEMS AND METHODS FOR GUIDED PLACEMENT OF ELECTROCARDIOGRAM ELECTRODES

(71) Applicant: KONINKLIJKE PHILIPS N.V., ENDHOVEN (NL)

(72) Inventors: Junmo An, Cambridge, MA (US); Richard Earl Gregg, Cambridge, MA (US)

(21) Appl. No.: 18/636,322

(22) Filed: Apr. 16, 2024

Related U.S. Application Data
(60) Provisional application No. 63/459,800, filed on Apr. 17, 2023.

(51) Int. Cl. A61B 5/00; A61B 5/28; A61B 90/00

(52) U.S. Cl. CPC A61B 5/6841; A61B 5/743; A61B 5/7435; A61B 90/08; A61B 5/28; A61B 2090/0807; A61B 2560/0487

(57) ABSTRACT

Electrocardiography (ECG or EKG) is a technique where electrodes are attached to the outer surface of the patient's skin at certain places on the patient's torso in order to monitor the electrical activity of the heart. The electrodes are connected by lead wires to an external device, which records the electrical activity of the heart over a period of time as detected by the electrodes and produces an electrocardiogram. Proper electrode placement is essential to measure the heart's electrical activity accurately and diagnose and interpret cardiac abnormalities. However, improper positioning or placement of the electrodes, including accidental interchanging of electrodes, are common technical mistakes that negatively impact signal quality and usefulness. Accordingly, provided herein are augmented-reality systems and methods of providing real-time guidance in placing one or more electrocardiogram electrodes on a subject and verification of such placements.

### CROSS-REFERENCE TO RELATED APPLICATIONS

This patent application claims the priority benefit under 35 U.S.C. § 119 (c) of U.S. Provisional Application No. 63/459,800, filed on Apr. 17, 2023, the contents of which are herein incorporated by reference.

### FIELD OF DISCLOSURE

The present disclosure relates generally to augmented-reality systems and methods, and more specifically to augmented-reality systems and methods adapted to provide real-time guidance and feedback during the placement of electrocardiogram electrodes.

### BACKGROUND

Electrocardiography (ECG or EKG) is a technique commonly used to record the heart's electrical activity through repeated cardiac cycles in order to monitor a patient's heartbeat for cardiac abnormalities. Employing this technique, several electrodes are attached to the outer surface of the skin at certain places on the patient's torso and extremities in order to monitor the electrical activity of the heart. These electrodes detect the small electrical changes that are a consequence of cardiac muscle depolarization followed by repolarization during each cardiac cycle. Changes in the normal ECG pattern occur in numerous cardiac conditions, including cardiac rhythm disturbances, such as atrial fibrillation and ventricular tachycardia, inadequate coronary artery blood flow, such as myocardial ischemia and myocardial infarction, and electrolyte disturbance, such as hypokalemia and hyperkalemia. The electrodes are connected by lead wires to an external device, which records the electrical activity of the heart over a period of time as detected by the electrodes. The data recording produced by the ECG technique is an electrocardiogram.

Conventional electrocardiograms employ ten electrodes for measuring the electrical activity of the heart, where each electrode is placed on a patient at a particular location within some tolerance. From these ten electrodes, twelve leads (i.e., potential differences) are measured and/or derived. For example, a right leg electrode (“RL”) can serve as a ground for the other electrodes, a first lead (Lead I) is measured from a right arm electrode (“RA”) to a left arm electrode (“LA”), a second lead (Lead II) is measured from the right arm electrode RA to a left leg electrode (“LL”), a third lead (Lead III) is measured from a left arm electrode LA to the left leg electrode LL, while the other nine leads include three augmented limb leads derived from combinations of Leads I-III and six chest leads derived from potential differences measured using six electrodes placed on the patient's chest at predetermined positions. Other conventional electrocardiograms include one, four, or five leads from a set of two, three, five or six electrodes. An electrocardiogram produced through these various arrangements is a valuable, non-invasive, diagnostic and monitoring tool that records the heart's electrical activity as waveforms and can detect various heart conditions.

Proper electrode placement is essential to measure the heart's electrical activity accurately and reliably diagnose and interpret cardiac abnormalities or arrhythmias because the standards for normal and abnormal conditions are based on those standard recordings. Improper positioning or placement of the electrodes, including accidental interchanging of electrodes, are common technical mistakes that negatively impact signal quality and diagnostics accuracy. Existing solutions require specialized training or dedicated assistive devices (e.g., glasses, garments, etc.) and do not address issues for the different numbers and various types of leads, and do not account for patients' age, sex, and body mass index (BMI). Further, these assistive devices may become easily lost or damaged and are oftentimes uncomfortable to wear.

### SUMMARY OF THE DISCLOSURE

Accordingly, the present disclosure relates to augmented-reality systems and methods of providing real-time guidance in placing one or more electrocardiogram electrodes on a subject that address drawbacks of conventional approaches.

According to an embodiment of the present disclosure, an augmented-reality system configured to provide real-time guidance in placing two or more electrocardiogram electrodes on a subject is provided. The augmented-reality system can include: at least one camera configured to generate visual data comprising one or more images; a display unit configured to display a visual feed comprising visual data and/or computer-augmented objects; one or more processors in communication with the display unit and the at least one camera; and a memory in communication with the one or more processors, the memory having stored thereon machine-readable instructions that, when executed by the one or more processors, cause the one or more processors to perform operations comprising: (i) receiving, via the at least one camera, a first visual data comprising at least one image showing at least a portion of the subject, wherein the portion of the subject includes the torso of the subject; (ii) analyzing the first visual data to generate a visual overlay template corresponding to the portion of the subject shown in the first visual data, wherein the visual overlay template includes recommended positions for one or more electrocardiogram electrodes; (iii) generating a composite visual feed based on the first visual data and the generated visual overlay template, wherein the visual overlay template is superimposed on the portion of the subject shown in the first visual data; and (iv) displaying, via the display unit, the composite visual feed.

In an aspect, the first visual data received from the at least one camera can include real-time images showing at least the portion of the subject, wherein the portion of the subject includes the torso of the subject.

In an aspect, the system further includes a user interface in communication with the one or more processors and configured to receive user input from an associated user, wherein the memory further includes machine-readable instructions stored thereon that, when executed by the one or more processors, cause the one or more processors to perform operations comprising: receiving, via the user interface, a user input indicating whether the generated visual overlay template accurately aligns with the portion of the subject as shown in the composite visual feed.

In an aspect, the memory further includes machine-readable instructions stored thereon that, when executed by the one or more processors, cause the one or more processors to perform operations comprising: generating instructions for placing one or more electrocardiogram electrodes on the torso of the subject if the user input indicates that the generated visual overlay template accurately aligns with the portion of the subject as shown in the composite visual feed; and generating instructions for adjusting either (i) a position of the at least one camera relative to the subject or (ii) one or more features of the generated visual overlay template if the user input indicates that the generated visual overlay template does not accurately align with the portion of the subject as shown in the composite visual feed. The memory can further include machine-readable instructions stored thereon that, when executed by the one or more processors, cause the one or more processors to perform operations comprising: receiving, via the user interface, a user input comprising one or more inputs for the subject, the inputs including at least one of an age of the subject, a gender of the subject, and a body mass index of the subject; wherein the visual overlay template is generated based on the first visual data received and the one or more inputs for the subject.

In an aspect, the visual overlay template includes either (i) a two-dimensional outline of a portion of a body that corresponds to the portion of the subject shown in the first visual data, or (ii) a three-dimensional model of a portion of a body that corresponds to the portion of the subject shown in the first visual data.

In an aspect, the memory further includes machine-readable instructions stored thereon that, when executed by the one or more processors, cause the one or more processors to perform operations comprising: receiving, via the at least one camera, a second visual data comprising at least one image showing one or more electrocardiogram electrodes placed on at least the portion of the subject; analyzing the second visual data to determine whether the one or more electrocardiogram electrodes shown in the second visual data are properly positioned; generating instructions for correcting the positioning of one or more of the electrocardiogram electrodes if it is determined that one or more of the electrocardiogram electrodes are not properly positioned; and generating a notification for a user associated with the system if it is determined that the one or more electrocardiogram electrodes are properly positioned, wherein the notification indicates that the one or more electrocardiogram electrodes are properly positioned.

In an aspect, the step of analyzing the second visual data includes: generating a projection for the subject based on the second visual data, wherein the projection includes expected positions of one or more electrocardiogram electrodes; registering a condition of each of the one or more electrocardiogram electrodes, wherein the condition of an electrocardiogram electrode includes a relative position of the electrocardiogram electrode and an identity of the electrocardiogram electrode; and comparing the projection with the registered conditions of each of the one or more electrocardiogram electrodes to determine whether the one or more electrocardiogram electrodes deviate from the expected positions.

According to another embodiment of the present disclosure, a non-transitory computer-readable storage medium having stored thereon machine-readable instructions is provided. When executed by one or more processors, the machine-readable instruction cause the one or more processors to perform operations comprising: (i) receiving, via at least one camera associated with the storage medium, a first visual data comprising at least one image showing at least a portion of the subject, wherein the portion of the subject includes the torso of the subject; (ii) analyzing the first visual data to generate a visual overlay template corresponding to the portion of the subject shown in the first visual data, wherein the visual overlay template includes recommended positions for one or more electrocardiogram electrodes; (iii) generating a composite visual feed based on the first visual data and the generated visual overlay template, wherein the visual overlay template is superimposed on the portion of the subject shown in the first visual data; and (iv) displaying, via a display unit associated with the storage medium, the composite visual feed.

According to yet another embodiment of the present disclosure, a computer-implemented method of providing real-time guidance in placing one or more electrocardiogram electrodes on a subject using an augmented-reality system that includes at least one camera, a display unit, one or more processors, and a memory having stored thereon machine-readable instructions is provided. The method can include: receiving, via the at least one camera, a first visual data comprising at least one image showing at least a portion of the subject, wherein the portion of the subject includes the torso of the subject; analyzing, via the one or more processors, the first visual data to generate a visual overlay template corresponding to the portion of the subject shown in the first visual data; generating, via the one or more processors, a composite visual feed based on the first visual data and the generated visual overlay template, wherein the visual overlay template is superimposed on the portion of the subject shown in the first visual data; and displaying, via the display unit, the composite visual feed.

In an aspect, the first visual data received from the at least one camera includes real-time images showing at least the portion of the subject, wherein the portion of the subject includes the torso of the subject.

In an aspect, the augmented-reality system further includes a user interface configured to receive user input form an associated user, and the method further comprises: receiving, via the user interface, a user input indicating whether the generated visual overlay template accurately aligns with the portion of the subject as shown in the composite visual feed.

In an aspect, the method further includes: generating instructions for placing one or more electrocardiogram electrodes on the torso of the subject if the user input indicates that the generated visual overlay template accurately aligns with the portion of the subject as shown in the composite visual feed.

In an aspect, the method further includes: generating instructions for adjusting either (i) a position of the at least one camera relative to the subject or (ii) one or more features of the generated visual overlay template if the user input indicates that the generated visual overlay template does not accurately align with the portion of the subject as shown in the composite visual feed.

In an aspect, the method further includes: receiving, via the at least one camera, a second visual data comprising at least one image showing one or more electrocardiogram electrodes placed on at least the portion of the subject; analyzing the second visual data to determine whether the one or more electrocardiogram electrodes shown in the second visual data are properly positioned; generating instructions for correcting the positioning of one or more of the electrocardiogram electrodes if it is determined that one or more of the electrocardiogram electrodes are not properly positioned; and generating a notification for a user associated with the system if it is determined that the one or more electrocardiogram electrodes are properly positioned, wherein the notification indicates that the one or more electrocardiogram electrodes are properly positioned.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a diagram illustrating the usage of an augmented-reality system configured to provide real-time guidance in placing one or more electrocardiogram electrodes on a subject according to aspects of the present disclosure.

FIG. 2A is a block diagram of an augmented reality system configured to provide real-time guidance in placing one or more electrocardiogram electrodes on a subject according to aspects of the present disclosure.

FIG. 2B is a block diagram of an augmented-reality user guidance package illustrated according to aspects of the present disclosure.

FIG. 3 is an illustration showing an exemplary user interface according to aspects of the present disclosure.

FIG. 4A is an illustration of an augmented-reality system according to aspects of the present disclosure.

FIG. 4B is an illustration showing the use of an augmented-reality system according to aspects of the present disclosure.

FIG. 4C is an illustration showing the use of an augmented reality system according to further aspects of the present disclosure.

FIG. 5 is an illustration showing electrode placement guidance provided by an augmented-reality system according to aspects of the present disclosure.

FIG. 6A is an illustration showing the verification of electrode placement using of an augmented-reality system according to aspects of the present disclosure.

FIG. 6B is an illustration showing the verification of electrode placement using of an augmented-reality system according to further aspects of the present disclosure.

FIG. 7 is a flowchart of a computer-implemented method of providing real-time guidance in placing one or more electrocardiogram electrodes on a subject using an augmented-reality system according to aspects of the present disclosure.

FIG. 8 is a flowchart of a computer-implemented process for configuring an augmented-reality system according to aspects of the present disclosure.

FIG. 9 is a flowchart of a computer-implemented method of providing real-time verification of electrode placement using an augmented-reality system according to aspects of the present disclosure.

### DETAILED DESCRIPTION OF EMBODIMENTS

The present disclosure is related to systems and methods of providing real-time guidance in the positioning of ECG electrodes and cables. As described herein, there are a number of common mistakes involved with the positioning of ECG electrodes and cables, even among healthcare professionals. For example, one of the most common mistakes includes the reversal or unintentional interchange of two lead-wires connected to the electrodes, such as the lead-wires connecting the right and left arm electrodes, the right arm and right leg electrodes, the left arm and left leg electrodes, and/or the right arm and left leg electrodes. Other common mistakes can include placing the chest electrodes (V1, V2, V3, V4, V5, and V6) too far away or too close, especially the distances between V1-V2 and V5-V6. Oftentimes, for example, certain electrodes (e.g., V1 and V2) may be placed too high, V5 and V6 may be placed too low, the chest electrodes may be laterally misplaced, or the chest lead wires may be unintentionally reversed. In further examples, the distance of the RA and LA electrodes may be too close to the patient's centerline or the height of the RL and LL electrodes on the torso may be too high. Each of these common mistakes can introduce variability to the ECG signals collected, which can negatively impact proper interpretation and diagnosis of cardiac function. Additionally, it is a challenge to compare electrocardiograms taken at different times when variations in ECG signal are introduced due to inconsistent placement of electrodes and lead-wires. Accordingly, the systems and methods of the present disclosure improve upon ECG measuring techniques by addressing these and other issues.

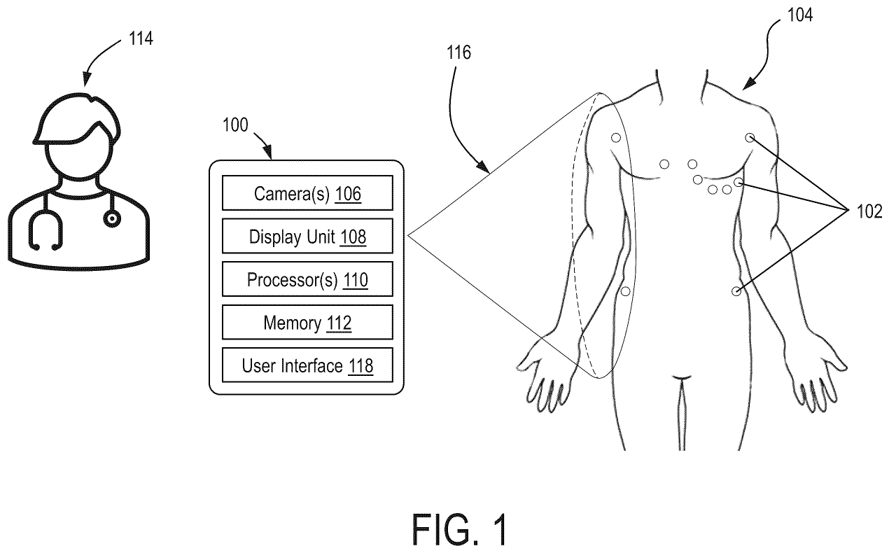

Turning to FIG. 1, an augmented-reality system 100 configured to provide real-time guidance in placing one or more electrocardiogram electrodes 102 on a subject 104 is illustrated in accordance with certain aspects of the present disclosure. As shown, the augmented-reality system 100 includes at least one camera 106, a display unit 108, one or more processors 110, and a memory 112 having machine-readable instructions stored thereon that, when executed by the one or more processors 110, cause the one or more processors 110 to perform one or more operations of the methods and/or processes described herein.

The at least one camera 106 may be configured to generate visual data comprising one or more images. The at least one camera 106 can have a fixed and/or adjustable a field of view 116 adequate to capture images showing at least a portion of the subject 104, such as the torso of the subject 104 as illustrated in FIG. 1. In embodiments, the at least one camera 106 can include one or more of digital imaging cameras, time of flight (ToF) cameras, depth cameras, LiDAR cameras, monochrome cameras, phase detection auto-focus cameras, infrared cameras, and/or the like.

The display unit 108 of the augmented-reality system 100 may be configured to display a visual feed comprising visual data obtained from the at least one camera 106 as well as one or more computer-augmented objects (e.g., computer-generated body models, etc.). In particular embodiments, the display unit 108 can be configured to display a composite visual feed comprising visual data obtained from the at least one camera 106 and one or more computer-augmented objects, as described in more detail below. In embodiments, the display unit 108 can be an internal display device, as in a mobile electronic device or a laptop device, or an external display device attached via an input/output interface, a head-mounted display, and/or a touchscreen.

As described herein, the one or more processors 110 may be in communication with the display unit 108 and the at least one camera 106. In embodiments, the one or more processors 110 are also in communication with the memory 112, which contains machine-readable instructions that, when executed by the one or more processors 110, cause the one or more processors 110 to provide real-time guidance in placing one or more electrocardiogram electrodes 102 on a subject 104. For example, in some embodiments, the memory 112 contains machine-readable instructions that, when executed by the one or more processors 110, cause the one or more processors 110 to perform operations comprising: (i) receiving, via the at least one camera 106, a first visual data comprising at least one image showing at least a portion of the subject 104, wherein the portion of the subject 104 includes the torso of the subject 104; (ii) analyzing the first visual data to generate a visual overlay template corresponding to the portion of the subject 104 shown in the first visual data, wherein the visual overlay template includes recommended positions for one or more electrocardiogram electrodes 102; (iii) generating a composite visual feed based on the first visual data and the generated visual overlay template, wherein the visual overlay template is superimposed on the portion of the subject 104 shown in the first visual data; and (iv) displaying, via the display unit 108, the composite visual feed.

In certain embodiments, the one or more processors 110 are in communication with the memory 112, which contains machine-readable instructions that, when executed by the one or more processors 110, cause the one or more processors 110 to verify the placement of one or more electrocardiogram electrodes on a subject 104 in real-time. For example, in embodiments, the memory 112 can contain machine-readable instructions that, when executed by the one or more processors 110, cause the one or more processors 110 to perform operations comprising: (i) receiving, via the at least one camera 106, a second visual data comprising at least one image showing one or more electrocardiogram electrodes 102 placed on at least the portion of the subject 104; (ii) analyzing the second visual data to determine whether the one or more electrocardiogram electrodes shown in the second visual data are properly positioned; (iii) generating instructions for correcting the positioning of one or more of the electrocardiogram electrodes 102 if it is determined that one or more of the electrocardiogram electrodes are not properly positioned; and (iv) generating a notification for a user 114 associated with the system 100 if it is determined that the one or more electrocardiogram electrodes 102 are properly positioned, wherein the notification indicates that the one or more electrocardiogram electrodes are properly positioned.

In embodiments, the augmented-reality system 100 may be associated with a user 114, which may be an individual other than the subject 104. In other embodiments, the subject 104 may also be the user 114 of the augmented-reality system 100. In particular embodiments, the augmented-reality system 100 or portions thereof can be embodied as an augmented reality-enabled device, such as an augmented reality-enabled personal computer, laptop computer, mobile device, smartphone, tablet computer, gaming device, consumer electronic device, headset, smart watch, and/or the like.

As shown in the example of FIG. 1, the augmented-reality system 100 can also include a user interface 118. The user interface 118 of the augmented-reality system 100 can be in communication with the one or more processors 110 and can be configured to generate instructions to present information to a user 114 associated with the augmented-reality system 100 and/or receive user input from an associated user 114. In embodiments, the user interface 118 includes hardware, software, or a combination of hardware and software. In embodiments, the hardware component of the user interface 118 can include one or more touch screen sensors, pressure sensors, fingerprint sensors, virtual or regular keyboards, a computer mouse, virtual or regular computer mice, touch pads, track pads, dials, and/or the like. In particular embodiments, the display unit 108 can also be the user interface 118 (i.e., in the case of a touch-enabled screen). As described in more detail below, the user input received via the user interface 118 can include combinations of text input, numerical input, and/or other forms of input responsive to one or more prompts of the augmented-reality system 100.

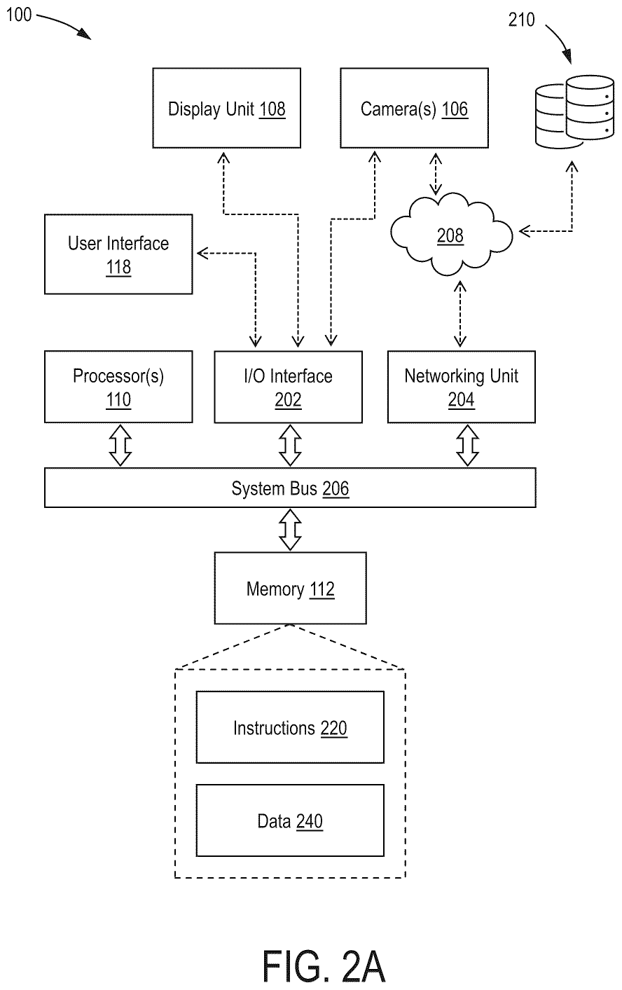

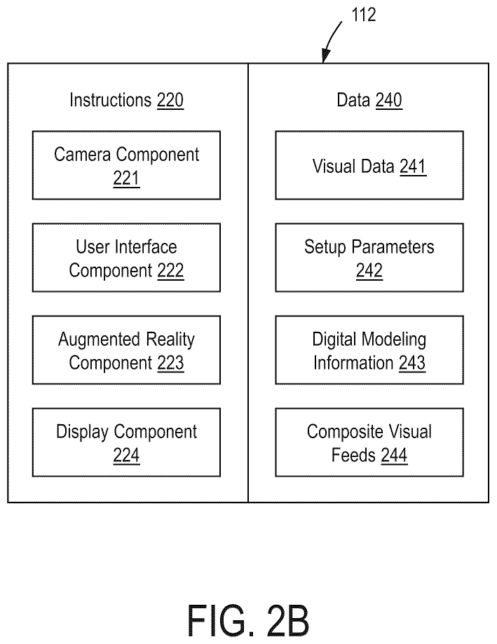

Turning to FIGS. 2A and 2B, these and other features of the augmented-reality systems of the present disclosure are described. In particular, as shown in FIG. 2A, an augmented-reality system 100 configured to provide real-time guidance in placing one or more electrocardiogram electrodes 102 on a subject 104 is illustrated. In embodiments, the augmented-reality system 100 comprises at least one camera 106, a display unit 108, one or more processors 110, machine-readable memory 112, and a user interface 118 as described above. In the example of FIG. 2A, the augmented-reality system 100 can also comprise an input/output (I/O) interface 202, a networking unit 204, and a system bus 206. As described herein, the different components of the augmented-reality system 100 can be interconnected and/or communicate through a system bus 206, which contains conductive circuit pathways through which instructions (e.g., machine-readable signals) and data may travel to effectuate communication, tasks, storage, and the like.

The one or more processors 110 may include one or more high-speed data processors adequate to execute the program components described herein and/or perform one or more steps of the methods described herein. The one or more processors 110 may include a microprocessor, a multi-core processor, a multithreaded processor, an ultra-low voltage processor, an embedded processor, and/or the like, including combinations thereof. The one or more processors 110 may include multiple processor cores on a single die and/or may be a part of a system on a chip (SoC) in which the processor 110 and other components are formed into a single integrated circuit, or a single package. In further examples, the one or more processors 110 can include specialize processors, such as one or more graphics processing units (GPUs). As a non-exhaustive list, the one or more processors 110 can include one or more of an Intel® Architecture Core™ based processor, such as a Quark™, an Atom™, an i3, an i5, and i7, or an MCU-class processor, an Advanced Micro Devices, Inc. (AMD) processor such as a Ryzen or Epyc based processor, an A5-A10 processor from Apple® Inc., a Snapdragon™ processor from Qualcomm® Technologies, Inc., a NVIDIA® GeForce RTX™, an AMD Radeon™ RX GPU, an Arm® Mali® GPU, and/or the like.

The I/O interface 202 of the augmented-reality system 100 may include one or more I/O ports that provide a physical connection to one or more devices, such as one or more cameras 106, display units 108, and/or user interfaces 118. Put another way, the I/O interface 202 may be configured to connect one or more peripheral devices of the augmented-reality system 100 in order to facilitate communication and/or control of between such devices. In some embodiments, the I/O interface 202 can comprise one or more serial ports.

The networking unit 204 of the augmented-reality system 100 may include one or more types of networking interfaces that facilitate wireless communication between one or more components of the augmented-reality system 100 and/or between the augmented-reality system 100 and one or more external components. In embodiments, the networking unit 204 may operatively connect the augmented-reality system 100 to a communications network (208), which can include a direct interconnection, the Internet, a local area network (“LAN”), a metropolitan area network (“MAN”), a wide area network (“WAN”), a wired or Ethernet connection, a wireless connection, a cellular network, and similar types of communications networks, including combinations thereof. In some examples, the augmented-reality system 100 may communicate with one or more remote/cloud-based servers 210 (including one or more cloud-based services), and/or wireless devices (e.g., such as a wireless camera 106) via the networking unit 204.

The memory 112 of the augmented-reality system 100 can be variously embodied in one or more forms of machine-accessible and machine-readable memory. In some examples, the memory 112 may comprise one or more types of memory, including one or more types of transitory and/or non-transitory memory. In particular embodiments, the memory 112 may be implemented as a magnetic disk storage device, an optical disk storage device, an array of storage devices, a solid-state memory device, and/or the like, including combinations thereof. The memory 112 may also include one or more other types of memory, such as dynamic random-access memory (DRAM), static random-access memory (SRAM), erasable programmable read-only memory (EPROM), electrically erasable programmable read-only memory (EEPROM), Flash memory, and/or the like.

As shown in the example of FIG. 2A, the memory 112 can be configured to store data 240 and machine-readable instructions 220 that, when executed by the one or more processors 110, cause the augmented-reality system 100 to perform one or more operations of the methods and/or processes described herein. The instructions 220 may include, but is not limited to, one or more software packages configured to perform one or more operations of the methods described herein. These software packages may be incorporated into, loaded from, loaded onto, or otherwise operatively available to the augmented-reality system 100.

With reference to FIG. 2B, according to certain embodiments of the present disclosure, the instructions 220 can include a camera component 221, a user interface component 222, an augmented-reality component 223, and/or a display component 224. Each of these components may include software, hardware, and/or some combination of both software and hardware.

In embodiments, the camera component 221 can be a stored program component that is executed by at least one processor, such as the one or more processors 110 of the augmented-reality system 100. In particular embodiments, the camera component 221 can be configured to operate one or more cameras associated with the augmented-reality system 100, such as the at least one camera 106. In embodiments, the camera component 221 can be configured to receive or otherwise generate visual data 241 comprising one or more images via the one or more cameras 106. As described herein, the visual data 241 can include one or more still-frame images in a digital, machine-readable format (e.g., RAW, JPEG, TIFF, and/or the like). In embodiments, the visual data 241 can also include one or more videos in a digital, machine-readable format (e.g., RAW, MP4 w/H.264, MP4 w/HEVC, XF-AVC, MOV, and/or the like). In embodiments, the visual data 241 can include a combination of still-frame images and videos.

In particular embodiments, the visual data 241 can include at least a first visual data comprising at least one image showing a subject 104 or a portion thereof. For example, in embodiments, the first visual data can include images or video of the subject 104, including images or video of the subject's torso (i.e., where the electrocardiogram electrodes 102 are to be placed). In embodiments, the first visual data may be captured prior to the placement of one or more electrocardiogram electrodes 102. As described herein, the first visual data can include real-time images or video of the subject 104, or can include still-frames or recorded video of the subject 104.

In further embodiments, the visual data 241 can include at least a second visual data comprising at least one image showing a subject 104 or a portion thereof after one or more electrocardiogram electrodes 102 have been placed. As mentioned above, the second visual data can include images or video of the subject 104, including images or video of the subject's torso where one or more electrocardiogram electrodes 102 are positioned.

In embodiments, the user interface component 222 can be a stored program component that is executed by at least one processor, such as the one or more processors 110 of the augmented-reality system 100. In particular embodiments, the user interface component 222 can be configured to generate instructions to present information to a user 114 of the augmented-reality system 100 and/or receive user input from the user 114. As described above, the user interface 118 can include hardware, software, or a combination of hardware and software. In embodiments, the software component of the user interface 118 can be an application that is stored on the augmented-reality system 100, or can be a web-based service that is accessible via a separate end-user device.

The user interface 118 generated by the user interface component 222 can be utilized to receive user input (e.g., via a hardware component of the augmented-reality system 100 as described above), and to present information, notifications, alerts, and/or the like to a user 114. For example, in particular embodiments, the user interface 118 can include one or more audio, visual, and/or haptic components that are utilized in conveying information, notifications, and/or alerts to the user 114.

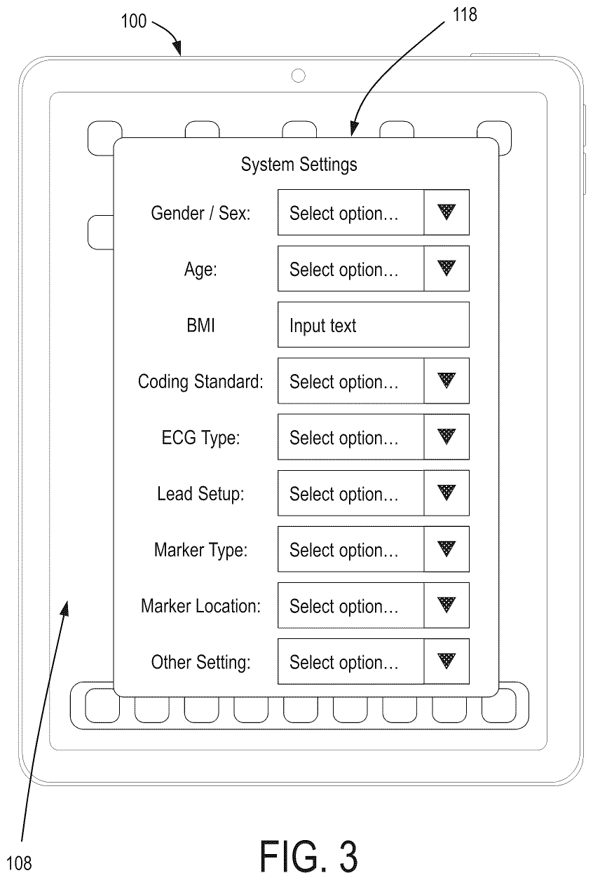

In further embodiments, the user interface component 222 can be configured to receive, via the user interface 118, user input that includes one or more setup parameters 242. In embodiments, these setup parameters 242 can be utilized by the augmented-reality system 100 to generate digital modeling information 243 for the subject 104 and verify the correct placement of the electrocardiogram electrodes 102. For example, according to various aspects of the present disclosure, the user input can include one or more of the following setup parameters 242: gender selection (e.g., male, female); age selection or input (e.g., neonates, infants, children, adolescents, adults, older adults, specific age); BMI or height and weight input (e.g., BMI, height, weight); color coding standard selection (e.g., International Electrotechnical Commission standard, American Heart Association standard); number of types of ECG leads selections (e.g., 12-lead ECG, 12-lead additions including left-sided posterior, right-sided posterior, right-sided anterior, at-home device; 5-lead ECG; 4-lead ECG; 1-lead ECG); marker type selection (e.g., a color-coded marker or a reflective passive marker); and/or marker location selection (e.g., xiphoid process and/or jugular notch). With reference to FIG. 3, one example of an augmented-reality system 100 having a display unit 108 and a user interface 118 configured to receive user input including one or more of the setup parameters 242 described above.

In embodiments, the augmented-reality component 223 can be a stored program component that is executed by at least one processor, such as the one or more processors 110 of the augmented-reality system 100. In particular embodiments, the augmented-reality component 223 can be configured to generate one or more composite visual feeds 244 based on a combination of the visual data 241 captured by the at least one camera 106 and digital modeling information 243.

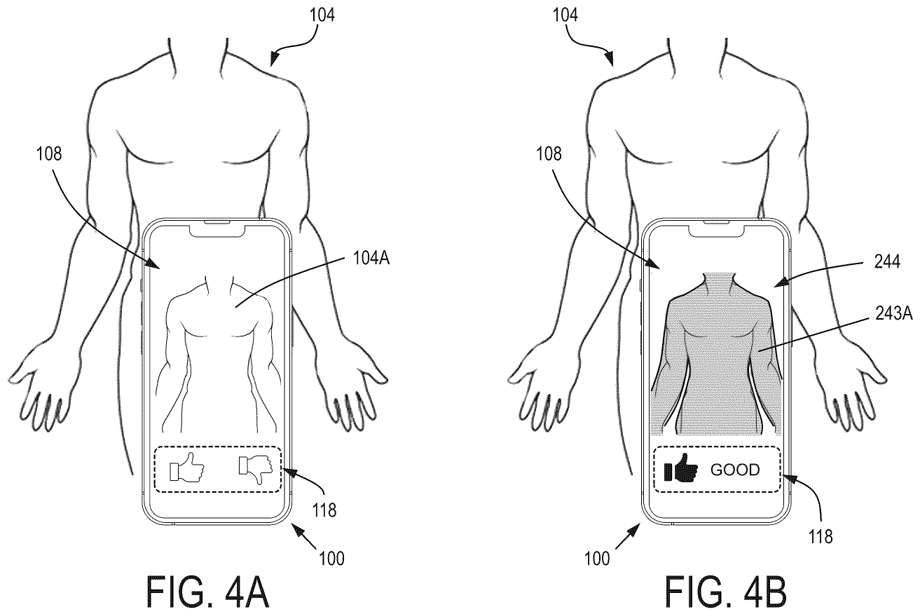

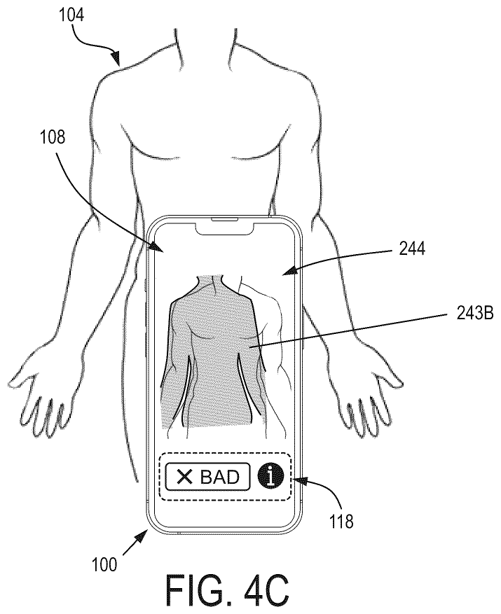

For example, with reference to FIGS. 4A, 4B, and 4C, the augmented-reality component 223 can be configured to generate at least a first composite visual feed 244 based on a combination of visual data 241 comprising images showing the torso of the subject 104 and digital modeling information 243 that includes a visual overlay template corresponding to the subject 104. In particular embodiments, if the visual data 241 includes the face of the subject 104, the composite visual feed 244 may de-identify the face of the subject 104, for example, by automatically detecting the face of the subject 104 and cropping or applying a blurring effect to the images of the visual data 241. In the example of FIG. 4A, a real-time visual 104A of the subject 104 is captured by at least one camera (not shown) of an augmented-reality system 100 and presented using a display unit 108. As shown in FIG. 4B, the augmented-reality component 223 has generated a composite visual feed 244 based on the visual data 241 and a visual overlay template 243A superimposed onto the real-time visual 104A of the subject 104.

In the example of FIG. 4B, the visual overlay template 243A accurately maps over the visual 104A of the subject 104 from the visual data. However, it is possible for the visual overlay template 243B to not accurately map over the visual 104A of the subject 104, as shown in the example of FIG. 4C. In such embodiments, the user interface 118 can include an option to provide an alert to the user 114 to correct the overlay 243A. Put another way, the augmented-reality component 223 can be configured to generate instructions for adjusting a position of the at least one camera 106 relative to the subject 104 such that a more appropriate visual of the torso can be obtained, or instructions for adjusting one or more features (e.g., size and/or setup parameters 242) of the overlay template 243B, if the visual overlay template 243B does not accurately align with the portion of the subject 104 as shown in the composite visual feed 244.

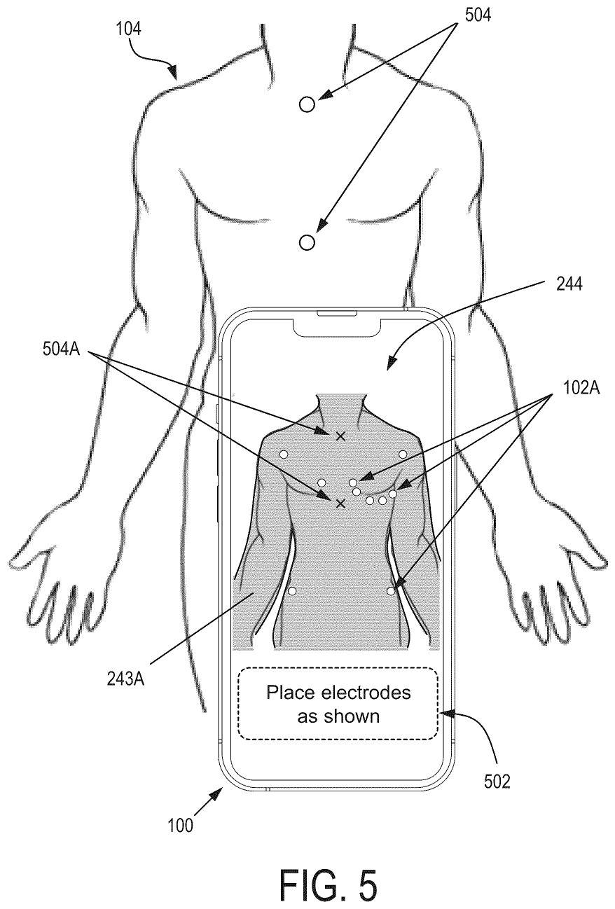

In embodiments where the visual overlay template 243A accurately aligns with the portion of the subject 104 in the composite visual feed 244 (as shown in FIG. 4B), the augmented-reality component 223 may generate instructions for placing one or more electrocardiogram electrodes 102 on the subject 104. For example, with reference to FIG. 5, the augmented-reality component 223 may generate a composite visual feed 244 that visually demonstrates where each of the one or more electrocardiogram electrodes 102A should be placed. The instructions generated by the augmented-reality component 223 may also include one or more textual, audible, and/or other forms of instructions, such as the instructions 502 shown in FIG. 5. In particular embodiments, the setup parameters 242 may define the configuration of electrodes 102 to be placed, and therefore the instructions generated for placing the electrodes 102 may be based on the user input (i.e., setup parameters 242). In further embodiments, the placement of the electrodes 102A as well as the alignment of the overlay template 243A may be coordinated using one or more physical markers 504 placed on the subject 104, which are they marked (i.e., using digital markers 504A) in the composite visual feed 244.

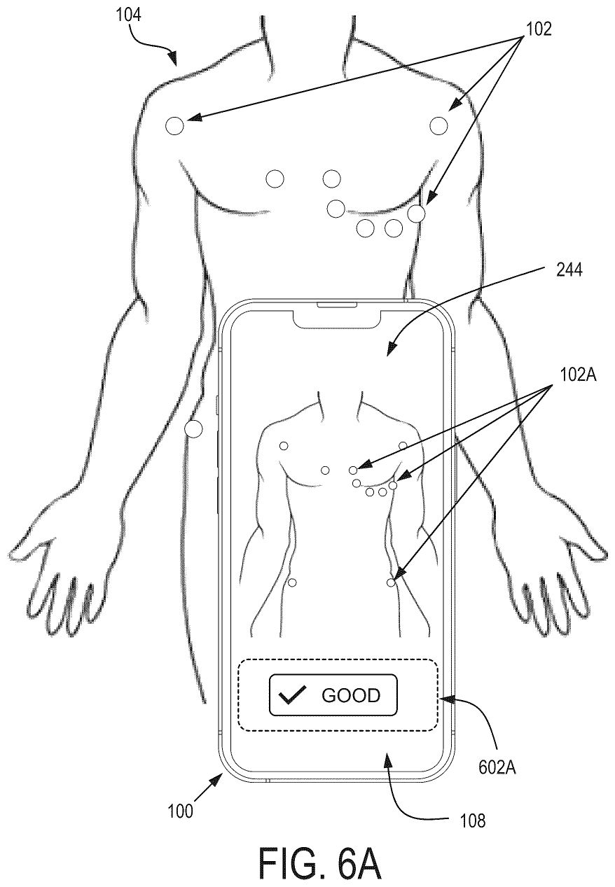

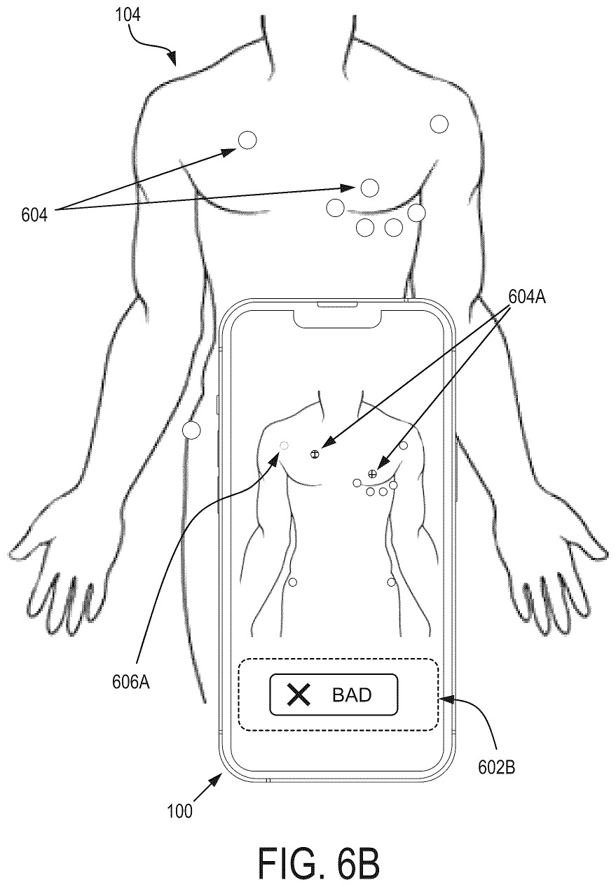

In embodiments, the augmented-reality component 223 may also be configured to determine whether one or more electrocardiogram electrodes 102 are properly positioned based on, for example, visual data 241 showing a subject 104 with one or more electrodes 102 and setup parameters 242 defining the proper positions of the one or more electrodes 102. For example, as shown in FIG. 6A, a second visual data 241 may be captured using the at least one camera 106 that includes one or more images showing one or more electrocardiogram electrodes 102 placed on the torso of the subject 104.

The augmented-reality component 223 is configured to analyze this visual data 241 to determine whether the one or more of the electrodes 102 are properly positioned by: (i) generating a projection for the subject showing the expected positions of the one or more electrodes 102A; registering a condition of the one or more electrodes 102, wherein the condition of an electrode 102 includes a relative position of the electrode 102 and an identity (e.g., RA, LA, RL, LL, V1, V2, etc.) of the electrode 102; and (iii) comparing the projected electrodes 102A with the registered conditions of each of the electrodes 102 to determine whether any of the electrodes 102 deviate from the projected electrodes 102A.

In embodiments, if it is determined that each of the electrocardiogram electrodes 102 are properly positioned, the augmented-reality system 100 can generate a notification for a user 114 indicating that the electrodes 102 are properly positioned. For example, as shown in FIG. 6A, the notification 602A indicating that the electrodes 102 are properly positioned may be displayed visually on the display unit 108.

In other embodiments, if it is determined that one or more of the electrocardiogram electrodes 102 are not properly positioned, the augmented-reality system 100 can generate a notification indicating incorrect placement and instructions for correcting the positioning of the improperly positioned electrodes. For example, as shown in FIG. 6B, electrodes 604 are identified as being improperly positioned by highlighting the projection 604A of these electrodes 604, and a missing electrode 606A is also identified. In such embodiments, the augmented-reality system 100 may generate a notification 602B indicating improper placement. In further embodiments, the augmented-reality system 100 may generate instructional directions for correcting the placement of the identified electrodes 604, 604A, 606A. In particular embodiments, these instructions can include, but are not limited to, one or more of the following: interchange right (RA) and left arm (LA) lead wires; interchange right arm (RA) and right left (RL) lead wires; interchange left arm (LA) and left leg (LL) lead wires, interchange right arm (RA) and left leg (LL) lead wires; V1 and/or V2 electrodes too high or too low; V1 and V2 electrodes are too far away from each other or too close to each other; V5 and V6 electrodes are too far away from each other or too close to each other; RA and LA electrodes are too close to each other; RL and LL electrodes are too high; and/or a lead-wire or electrodes is missing.

As described herein, one or more of the composite visual feeds 244 may be static, meaning that a single still image from a set of visual data 241 is utilized and displayed. In such embodiments, the relative position and orientation of the subject 104 and/or electrodes 102 may be determined once, and the corresponding modeling information 243 (e.g., projections, overlay templates, etc.) is superimposed onto the static image. In other embodiments, the composite visual feed 244 may be dynamic, such as when the visual feed 244 includes a real-time video of subject 104. In such embodiments, the modeling information 243 (e.g., projections, overlay templates, etc.) may be updated based on changes in the relative position and orientation of the subject 104 within the visual data 241.

In embodiments, the display component 224 can be a stored program component that is executed by at least one processor, such as the one or more processors 110 of the augmented-reality system 100. In particular embodiments, the display component 224 can be configured to operate the display unit 108, including being able to cause the generated composite visual feeds 244 and notifications/instructions 502, 602A, 602B to be displayed at the appropriate times.

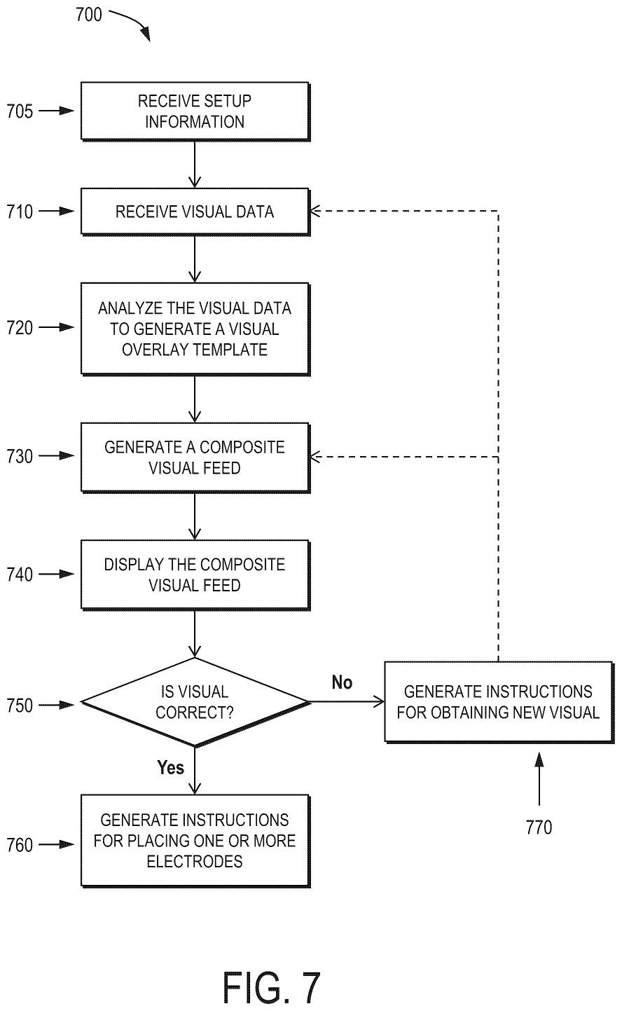

Also provided herein are computer-implemented methods of providing real-time guidance in placing one or more electrocardiogram electrodes on a subject using an augmented-reality system (e.g., system 100). For example, with reference to FIG. 7, a computer-implemented method 700 of providing real-time guidance in placing one or more electrocardiogram electrodes on a subject using an augmented-reality system is illustrated according to certain aspects of the present disclosure. As shown, the method 700 includes: in a step 710, receiving a first visual data comprising at least one image showing at least a portion of a subject using at least one camera (e.g., camera 106) of an augmented-reality system (e.g., system 100), wherein the portion of the subject includes the subject's torso; in a step 720, analyzing the first visual data to generate a visual overlay template corresponding to the portion of the subject shown in the first visual data; in a step 730, generating a composite visual feed based on the first visual data and the generated visual overlay template, wherein the visual overlay template is superimposed on the portion of the subject (i.e., the subject's torso) captured in the first visual data; in a step 740, displaying the composite visual feed via a display unit (e.g., display unit 108) of the augmented-reality system.

At the step 710, the method 700 includes receiving visual data that comprises one or more images of the subject, including one or more images of the subject's torso (i.e., where the electrodes 102 are to be placed). As described above, the visual data can include still frame images or can include real-time video of the subject that is captured using at least one camera (e.g., camera 106) of an augmented-reality system (e.g., system 100).

At the step 720, the method 700 includes analyzing the visual data to generate a visual overlay template corresponding to the subject. In embodiments, the step 720 can include estimating a distance of the camera (e.g., camera 106) from the subject (e.g., subject 104), estimating a two-dimensional or three-dimensional pose or position of the subject (e.g., standing up, straight, laying down, sitting in a chair, etc.) from visuals using deep learning based human pose estimation approaches, estimating an angle of the camera relative to the subject or the ground, estimating an angle of the camera relative to the subject based on different focal points, building a projection of the subject that accounts for the relative angle/perspective of the camera, estimating the BMI, and cycling through BMI models and adjusting the model height to find the best fit to the images of the subject.

According to various aspects of the present disclosure, the visual overlay template can be generated using two-dimension human pose estimation (i.e., by estimating the two-dimensional position or spatial location of human body key points from visuals) using applications such as OpenPose, CPN, AlphaPose, HRNet, DCPose, and/or similar tools. In further embodiments, the visual overlay template can be generated using three-dimensional human pose estimation (i.e., by predicting the locations of body joints in three-dimensional space) using applications such as OpenPose and/or similar tools. As described above, one or more of these tools may also be used for tracking the motion of the subject and adjusting the overlay template in real-time based on the movement of the subject.

At the step 730, the method 700 includes generating a composite visual feed based on the visual data captured and the visual overlay template, wherein the visual overlay template is superimposed onto the images of the subject.

At the step 740, the method 700 includes displaying the composite visual feed using a display unit of the augmented-reality system. For example, as shown in FIGS. 4B and 4C, the composite visual feed may be displayed on a display screen of an augmented-reality system, which may be embodied as a mobile electronic device.

At the step 750, the method 700 includes receiving user input indicating whether the generated visual overlay template accurately aligns with the subject as shown in the composite visual feed. The user input may be received via a user interface, which may be variously embodied as described above. In particular embodiments, if the visual overlay template accurately aligns with the subject as shown in FIG. 4B, for example, a user (e.g., user 114) may input feedback indicating that the method 700 should proceed to the next step. In other embodiments, if the visual overlay template does not accurately align with the subject as shown in FIG. 4C, the user (e.g., user 114) may input feedback indicating that adjustments need to be made before providing electrode placement guidance. In some embodiments, the user input may change one or more parameters used to generate the visual overlay template, such as an estimated BMI value.

For example, at the step 760, the method 700 includes generating instructions for placing one or more electrodes onto the subject once confirmation of the visual overlay template is received. Alternatively, if confirmation of the visual overlay template is not received, then the method 700 can include, in a step 770, generating instructions for obtaining a new visual or otherwise adjusting the visual overlay template. In particular embodiments, the step 770 can include adjusting the position of the camera relative to the subject such that a better view of the subject is obtained, or adjusting one or more features of the visual overlay template to better match with the images of the subject in real-time.

Additionally, as shown in FIG. 7, the method 700 can optionally include, in a step 705, receiving setup information for the subject (e.g., subject 104) via a user interface of the augmented-reality system (e.g., system 100). In particular embodiments, the setup information can include archived data associated with a specific patient that is retrieved and loaded from a patient profile in order to generate the visual overlay template. In other embodiments, the setup information can include one or more setup parameters received via a user interface (as shown in FIG. 3), including but not limited to: gender selection (e.g., male, female); age selection or input (e.g., neonates, infants, children, adolescents, adults, older adults, specific age); BMI or height and weight input (e.g., BMI, height, weight); color coding standard selection (e.g., International Electrotechnical Commission standard, American Heart Association standard); number of types of ECG leads selections (e.g., 12-lead ECG, 12-lead additions including left-sided posterior, right-sided posterior, right-sided anterior, at-home device; 5-lead ECG; 4-lead ECG; 1-lead ECG); marker type selection (e.g., a color-coded marker or a reflective passive marker); and/or marker location selection (e.g., xiphoid process and/or jugular notch).

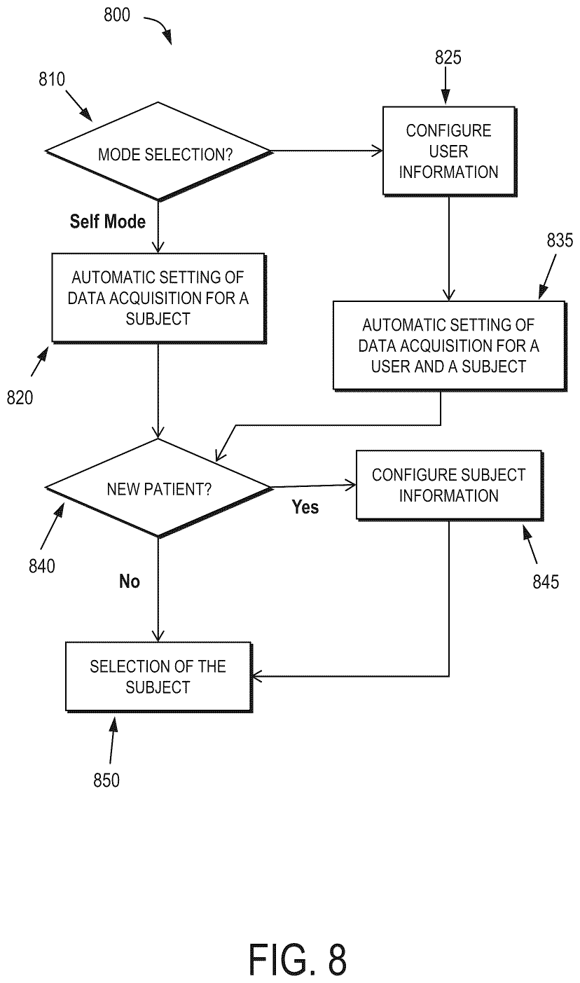

More specifically, the step 705 can include the process 800 illustrated in FIG. 8, which includes: in an operation 810, receiving or determining a mode selection; in an operation 820, automatically setting data acquisition for the subject if a self-mode is selected; in an operation 825, configuring user information if a user mode is selected; in an operation 835, automatically setting data acquisition for the user and the subject; in an operation 840, receiving or determining whether the subject is a new patient; and in an operation 845, configuring subject information if the subject is a new patient. In embodiments, the process 800 then includes, in an operation 850, selection of the subject, which can include receiving visual data of the subject and analyzing the visual data (e.g., steps 710, 720) as described above.

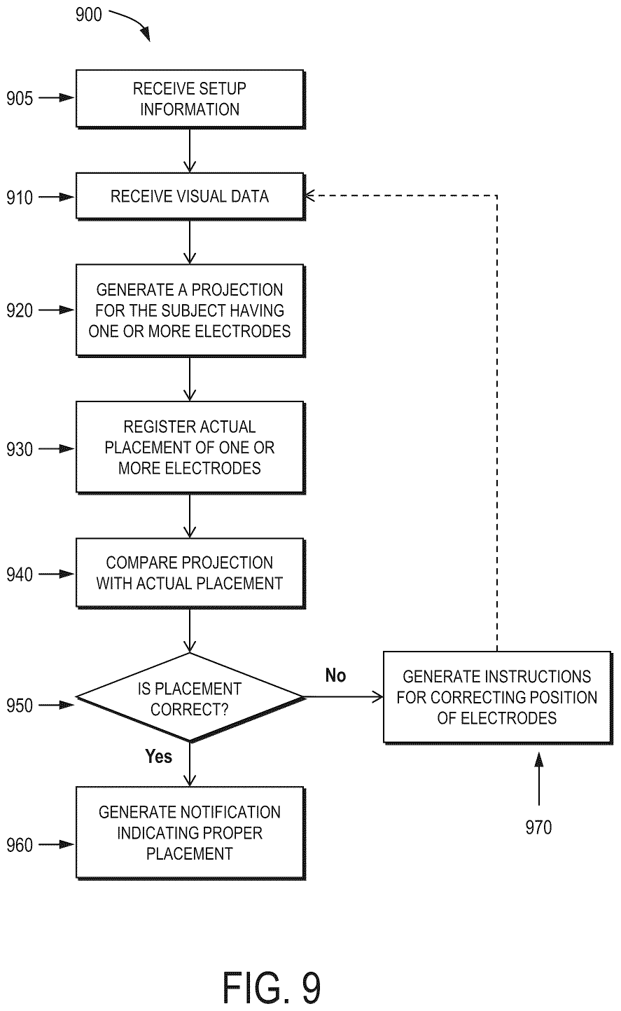

Also provided herein are computer-implemented methods of providing real-time verification of electrocardiogram electrode placement using an augmented-reality system (e.g., system 100). For example, with reference to FIG. 9, a computer-implemented method 900 of providing real-time verification of electrocardiogram electrode placement using an augmented-reality system is illustrated according to certain aspects of the present disclosure. As shown, the method 900 includes: in a step 910, receiving visual data comprising at least one image showing one or more electrocardiogram electrodes placed on at least a portion of a subject (i.e., the torso of a subject); in one or more step 920-950, analyzing the visual data to determine whether the electrodes are properly positioned; in a step 960, generating a notification to a user associated with the system if the electrodes are properly positioned, wherein the notification indicates that the electrodes are properly positioned; and in a step 970, generating instructions for correcting the position of one or more electrodes if it is determined that one or more electrodes are improperly positioned.

At the step 910, the method 900 includes receiving visual data that comprises one or more images of the subject, including one or more images of the subject's torso (i.e., where the electrodes 102 are to be placed). As described above, the visual data can include a single picture taken of the subject after the placement of the ECG electrodes and cables, or can include a video of the subject taken after placement of the ECG electrodes and cables.

In one or more steps (e.g., steps 920-950), the method 900 includes analyzing the visual data to determine whether the electrodes are properly positioned. As shown in FIG. 9, this operation can include: in a step 920, generating a projection for the subject that includes the expected positions of one or more electrocardiogram electrodes; in a step 930, registering a condition of each of the one or more electrodes placed on the subject; in a step 940, comparing the projection with the expected positions of the electrodes to the actual placement of electrodes; and, in a step 950, determining whether the electrodes are properly positioned.

More specifically, in the step 920, the method 900 can include generating a two-dimensional or three-dimensional projection for the subject based on the various setup parameters and the visual data, wherein the projection includes the expected location of one or more electrodes. In some embodiments, the projection can also include a translucent rib cage which helps to reinforce where electrodes go compared to anatomical landmarks, as well as one or more dotted midlines sectioning the torso of the subject. In embodiments, the projection can be based on the setup parameters described herein. For example, if a 5-lead setup is selected, then the projection will include a corresponding number of electrodes and lead wires. Similarly, the projection may be adjusted based on the height/weight or BMI parameters received, as well as the position or orientation of the subject as shown in the visual data, for example.

In embodiments, the step 920 can include estimating a distance of the camera (e.g., camera 106) from the subject (e.g., subject 104), estimating a two-dimensional or three-dimensional pose or position of the subject (e.g., standing up, straight, laying down, sitting in a chair, etc.) from visuals using deep learning based human pose estimation approaches, estimating an angle of the camera relative to the subject or the ground, estimating an angle of the camera relative to the subject based on different focal points, building a projection of the subject that accounts for the relative angle/perspective of the camera, and cycling through BMI models and adjusting the model height to find the best fit to the images of the subject.

In particular embodiments, the projection can be generated using two-dimension human pose estimation (i.e., by estimating the two-dimensional position or spatial location of human body key points from visuals) using applications such as OpenPose, CPN, AlphaPose, HRNet, DCPose, and/or similar tools. In further embodiments, the projection can be generated using three-dimensional human pose estimation (i.e., by predicting the locations of body joints in three-dimensional space) using applications such as OpenPose and/or similar tools. As described above, one or more of these tools may also be used for tracking the motion of the subject and adjusting the projection in real-time based on the movement of the subject.

In the step 930, the method 900 can include registering that actual placement and/or identity of one or more electrodes on the subject by analyzing the visual data. Put another way, the visual data may be analyzed to determine a relative location of each electrode and identify whether each electrode was placed in the correct spot. As described above, the user input may define a particular color-coding scheme and use that color-coding scheme to determine whether each electrode was placed in the correct spot.

In the step 940 and 950, the method 900 includes comparing the projection with the actual placement of the electrodes. If all of the electrodes are correctly positioned based on the setup information received, then the method 900 can include, in a step 960, generating a notification to the user (e.g., user 114) indicating that the electrodes were properly placed. In other embodiments, if one or more electrodes are improperly placed or missing, the method 900 can include, in a step 970, generating instructions for correcting the position of the electrodes or lead wires. In particular embodiments, these instructions can include, but are not limited to, one or more of the following: interchange right (RA) and left arm (LA) lead wires; interchange right arm (RA) and right left (RL) lead wires; interchange left arm (LA) and left leg (LL) lead wires, interchange right arm (RA) and left leg (LL) lead wires; move V1 and/or V2 electrodes higher or lower; move V1 and V2 electrodes closer together or farther apart; move V5 and V6 electrodes closer together or farther apart; move RA and LA electrodes farther apart; move RL and LL electrodes lower; and/or a lead wire or electrodes is missing.

Additionally, as shown in FIG. 9, the method 900 can optionally include, in a step 905, receiving setup information for the subject (e.g., subject 104) via a user interface of the augmented-reality system (e.g., system 100). In particular embodiments, the setup information can include archived data associated with a specific patient that is retrieved and loaded from a patient profile in order to generate the visual overlay template. In other embodiments, the setup information can include one or more setup parameters received via a user interface (as shown in FIG. 3), including but not limited to: gender selection (e.g., male, female); age selection or input (e.g., neonates, infants, children, adolescents, adults, older adults, specific age); BMI or height and weight input (e.g., BMI, height, weight); color coding standard selection (e.g., International Electrotechnical Commission standard, American Heart Association standard); number of types of ECG leads selections (e.g., 12-lead ECG, 12-lead additions including left-sided posterior, right-sided posterior, right-sided anterior, at-home device; 5-lead ECG; 4-lead ECG; 1-lead ECG); marker type selection (e.g., a color-coded marker or a reflective passive marker); and/or marker location selection (e.g., xiphoid process and/or jugular notch).

More specifically, the step 905 can include the process 800 illustrated in FIG. 8, which includes: in an operation 810, receiving or determining a mode selection; in an operation 820, automatically setting data acquisition for the subject if a self-mode is selected; in an operation 825, configuring user information if a user mode is selected; in an operation 835, automatically setting data acquisition for the user and the subject; in an operation 840, receiving or determining whether the subject is a new patient; and in an operation 845, configuring subject information if the subject is a new patient. In embodiments, the process 800 then includes, in an operation 850, selection of the subject, which can include receiving visual data of the subject and analyzing the visual data (e.g., steps 910-950) as described above.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. An augmented-reality system configured to provide real-time guidance in placing one or more electrocardiogram electrodes on a subject, the system comprising:
at least one camera configured to generate visual data comprising one or more images;
a display unit configured to display a visual feed comprising visual data and/or computer-augmented objects;
one or more processors in communication with the display unit and the at least one camera; and
a memory in communication with the one or more processors, the memory having stored thereon machine-readable instructions that, when executed by the one or more processors, cause the one or more processors to perform operations comprising:
(i) receiving, via the at least one camera, a first visual data comprising at least one image showing at least a portion of the subject, wherein the portion of the subject includes a torso of the subject;
(ii) analyzing the first visual data to generate a visual overlay template corresponding to the portion of the subject shown in the first visual data, wherein the visual overlay template includes recommended positions for one or more electrocardiogram electrodes;
(iii) generating a composite visual feed based on the first visual data and the generated visual overlay template, wherein the visual overlay template is superimposed on the portion of the subject shown in the first visual data; and
(iv) displaying, via the display unit, the composite visual feed.

2. The augmented-reality system of claim 1, wherein the first visual data received from the at least one camera comprises real-time images showing at least the portion of the subject, wherein the portion of the subject includes the torso of the subject.

3. The augmented-reality system of claim 1, further comprising:
a user interface in communication with the one or more processors and configured to receive user input from an associated user,
wherein the memory further includes machine-readable instructions stored thereon that, when executed by the one or more processors, cause the one or more processors to perform operations comprising:
receiving, via the user interface, a user input indicating whether the generated visual overlay template accurately aligns with the portion of the subject as shown in the composite visual feed.

4. The augmented-reality system of claim 3, wherein the memory further includes machine-readable instructions stored thereon that, when executed by the one or more processors, cause the one or more processors to perform operations comprising:
generating instructions for placing one or more electrocardiogram electrodes on the torso of the subject if the user input indicates that the generated visual overlay template accurately aligns with the portion of the subject as shown in the composite visual feed; and
generating instructions for adjusting either (i) a position of the at least one camera relative to the subject or (ii) one or more features of the generated visual overlay template if the user input indicates that the generated visual overlay template does not accurately align with the portion of the subject as shown in the composite visual feed.

5. The augmented-reality system of claim 3, wherein the memory further includes machine-readable instructions stored thereon that, when executed by the one or more processors, cause the one or more processors to perform operations comprising:
receiving, via the user interface, a user input comprising one or more inputs for the subject, the inputs including at least one of an age of the subject, a gender of the subject, and a body mass index of the subject;
wherein the visual overlay template is generated based on the first visual data received and the one or more inputs for the subject.

6. The augmented-reality system of claim 1, wherein the visual overlay template comprises either (i) a two-dimensional outline of a portion of a body that corresponds to the portion of the subject shown in the first visual data, or (ii) a three-dimensional model of a portion of a body that corresponds to the portion of the subject shown in the first visual data.

7. The augmented-reality system of claim 1, wherein the memory further includes machine-readable instructions stored thereon that, when executed by the one or more processors, cause the one or more processors to perform operations comprising:
receiving, via the at least one camera, a second visual data comprising at least one image showing one or more electrocardiogram electrodes placed on at least the portion of the subject;
analyzing the second visual data to determine whether the one or more electrocardiogram electrodes shown in the second visual data are properly positioned;
generating instructions for correcting the positioning of one or more of the electrocardiogram electrodes if it is determined that one or more of the electrocardiogram electrodes are not properly positioned; and
generating a notification for a user associated with the system if it is determined that the one or more electrocardiogram electrodes are properly positioned, wherein the notification indicates that the one or more electrocardiogram electrodes are properly positioned.

8. The augmented-reality system of claim 7, wherein the step of analyzing the second visual data includes:
generating a projection for the subject based on the second visual data, wherein the projection includes expected positions of one or more electrocardiogram electrodes;
registering a condition of each of the one or more electrocardiogram electrodes, wherein the condition of an electrocardiogram electrode includes a relative position of the electrocardiogram electrode and an identity of the electrocardiogram electrode; and
comparing the projection with the registered conditions of each of the one or more electrocardiogram electrodes to determine whether the one or more electrocardiogram electrodes deviate from the expected positions.

9. A non-transitory computer-readable storage medium having stored thereon machine-readable instructions that, when executed by one or more processors, cause the one or more processors to perform operations comprising:
(i) receiving, via at least one camera associated with the storage medium, a first visual data comprising at least one image showing at least a portion of a subject, wherein the portion of the subject includes a torso of the subject;
(ii) analyzing the first visual data to generate a visual overlay template corresponding to the portion of the subject shown in the first visual data, wherein the visual overlay template includes recommended positions for one or more electrocardiogram electrodes;
(iii) generating a composite visual feed based on the first visual data and the generated visual overlay template, wherein the visual overlay template is superimposed on the portion of the subject shown in the first visual data; and
(iv) displaying, via a display unit associated with the storage medium, the composite visual feed.

10. A computer-implemented method of providing real-time guidance in placing one or more electrocardiogram electrodes on a subject using an augmented-reality system that includes at least one camera, a display unit, one or more processors, and a memory having stored thereon machine-readable instructions, the method comprising:
receiving, via the at least one camera, a first visual data comprising at least one image showing at least a portion of the subject, wherein the portion of the subject includes a torso of the subject;
analyzing, via the one or more processors, the first visual data to generate a visual overlay template corresponding to the portion of the subject shown in the first visual data;
generating, via the one or more processors, a composite visual feed based on the first visual data and the generated visual overlay template, wherein the visual overlay template is superimposed on the portion of the subject shown in the first visual data; and
displaying, via the display unit, the composite visual feed.

11. The computer-implemented method of claim 10, wherein the first visual data received from the at least one camera comprise real-time images showing at least the portion of the subject, wherein the portion of the subject includes the torso of the subject.

12. The computer-implemented method of claim 10, wherein the augmented-reality system further comprises a user interface configured to receive user input form an associated user, and the method further comprises:
receiving, via the user interface, a user input indicating whether the generated visual overlay template accurately aligns with the portion of the subject as shown in the composite visual feed.

13. The computer-implemented method of claim 12, further comprising:
generating instructions for placing one or more electrocardiogram electrodes on the torso of the subject if the user input indicates that the generated visual overlay template accurately aligns with the portion of the subject as shown in the composite visual feed.

14. The computer-implemented method of claim 12, further comprising:
generating instructions for adjusting either (i) a position of the at least one camera relative to the subject or (ii) one or more features of the generated visual overlay template if the user input indicates that the generated visual overlay template does not accurately align with the portion of the subject as shown in the composite visual feed.

15. The computer-implemented method of claim 10, further comprising:
receiving, via the at least one camera, a second visual data comprising at least one image showing one or more electrocardiogram electrodes placed on at least the portion of the subject;
analyzing the second visual data to determine whether the one or more electrocardiogram electrodes shown in the second visual data are properly positioned;
generating instructions for correcting the positioning of one or more of the electrocardiogram electrodes if it is determined that one or more of the electrocardiogram electrodes are not properly positioned; and
generating a notification for a user associated with the system if it is determined that the one or more electrocardiogram electrodes are properly positioned, wherein the notification indicates that the one or more electrocardiogram electrodes are properly positioned.
